(19) United States
(12) Patent Application Publication
(10) Pub. No.: US 2004/0198794 A1
(43) Pub. Date: Oct. 7, 2004

(54) Ondansetron forms and processes of making the same

(76) Inventors: Raymond J. H. Westheim, Nijmegen (NL); Frans van Dalen, Nijmegen (NL)

Correspondence Address: MARK R. BUSCHER, P.O. BOX 161, CATHARPIN, VA, 20143, US

(21) Appl. No.: 10/750,211

(22) Filed: Jan. 2, 2004

Related U.S. Application Data
(60) Provisional application No. 60/438,780, filed on Jan. 9, 2003.

(51) Int. Cl. A61K 31/4178; C 07D 4 3/02

(52) U.S. Cl. 514/397; 548/311.4

(57) ABSTRACT

Ondansetron forms are identified and processes for making the same are provided. The ondansetron can be used in various pharmaceutical applications.

This application claims the benefit under 35 U.S.C. § 119(e) from U.S. provisional patent application Serial No. 60/438,780, filed Jan. 9, 2003, the entire contents of which are incorporated herein.

### BACKGROUND OF THE INVENTION

The present invention is directed to solid state forms of ondansetron base and methods for making various forms.

Ondansetron is a pharmaceutically active agent commonly used for the treatment of nausea and vomiting, particularly when associated with cancer chemotherapy treatments. In marketed compositions (sold under brand name ZOFRAN® by Glaxo), ondansetron is used as a free base in rapidly dissolvable tablets and as a hydrochloride salt in injections, tablets for oral administration and oral solutions. Ondansetron is chemically named 1,2,3,9-tetrahydro-9-methyl-3-((2-methyl-1H-imidazol-1 yl)methyl-4H-carbazol-4-one and has the following chemical structure:

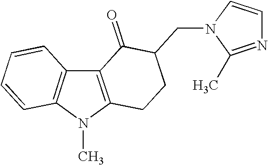

Because the ondansetron molecule has one optically active carbon, it can exist as two different enantiomers or as a mixture thereof, i.e., as a racemate. Both enantiomers are pharmaceutically active, however only the racemate is marketed thus far.

DE 3502508 and corresponding U.S. Pat. No. 4,695,578 describe ondansetron and various other 3-imidazole-tetrahydrocarbazolones, as useful in the treatment of migraine and psychotic disorders such as schizophrenia. The U.S. Pat. No. 4,695,578 discloses several synthetic routes for making ondansetron. One example uses a transamination reaction as shown below:

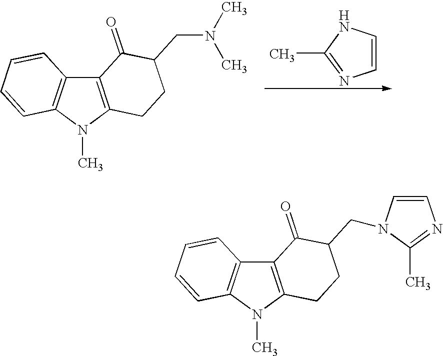

wherein an aqueous solution of 3-((dimethylamino)methyl)-1,2,3,9-tetrahydro-9-methyl-4H-carbazol-4-one hydrochloride is treated with 2-methylimidazole and heated at reflux for twenty hours. The crude ondansetron base is reported (Example 4) to have a maximum melting point of 224° C. while the product recrystallized from methanol has a melting point of about 231-232° C. (Example 7) or 232-234° C. (Example 8) under decomposition. Ondansetron base obtained after treatment of the reaction mixture by a column chromatography gave a product of melting point 228-229° C. (Example 18). Other than the melting point property, which differs from example to example, little information is given regarding the solid state material.

Ondansetron base prepared by other methods have reported various melting points from 215° C. up to 228.5° C. For example:

| | Ondansetron Melting Point |
|---|---|
| Patent | (Max or range) |
| EP 595111/U.S. Pat. No. 5478949 | 225° C. |
| EP 221629/U.S. Pat. No. 4957609 | 215-216 C. |
| EP 219929/U.S. Pat. No. 4739072 | 227.5-228.5 C. |

In EP 595111/U.S. Pat. No. 5,478,949, the purity is noted as 97.6%. In EP 219929/U.S. Pat. No. 4,739,072 the ondansetron base was reported to contain 0.31 mol % of water, which corresponds to 1.87% water by weight.

It is apparent that the reported data of melting points are different and it is difficult to judge the reason for the variations. It is generally known that the melting point of a solid material may be affected by the purity of the substance (the impurities tend to decrease the melting temperature) and it is also known that presence of trace contaminants may affect the formation and properties of crystalline lattice of the solid compound, resulting in changes in the crystalline forms and solid state properties (solubility, colour etc.). The thermodynamic and kinetic aspects associated with conditions of solid state formation (e.g., temperature of crystal formation, rate of cooling, concentration and kind of the solvent, etc.) may also contribute to the differences, as one may isolate two solid state materials by different techniques that are chemically identical but have different crystalline structure. The crystal structure of the ondansetron base is not set forth in any of the above-mentioned patents and thus it is unclear if the variation in melting point is due to impurities, measuring techniques, or polymorphic structure.

It would be desirable to identify and isolate additional forms of ondansetron. Further, it would be desirable to have reliable processes for producing ondansetron in one or more forms.

### SUMMARY OF THE INVENTION

The present invention is based on the discovery of various forms of ondansetron and processes for making the same. Accordingly, a first aspect of the invention relates to a solid crystalline ondansetron having at least one of the following characteristics:

a DSC melting endotherm peak greater than or equal to 240° C.;

a trace amount of a base or residue thereof comprising an alkali metal, an amine, an ammonium, or an ion thereof; or

a water content of 1.3 to 1.5 wt %.

The ondansetron solid form having a melting endotherm peak of at least 240° C., typically has a peak within the range of 240° C. to 255° C. and preferably has a first melting endotherm peak within the range of 240° C. to 249° C. and frequently has a second, higher endotherm peak, typically between 249° C. and 255° C. The ondansetron having a trace amount of a base or residue preferably contains 1 ppm to 1000 ppm of the base or residue. The base or residue is normally provided in the crystal structure by a neutralization process, which is described hereinafter, although such is not required. Preferably the base or residue comprises sodium or a sodium ion. The ondansetron forms can be anhydrous or hydrated. However, a preferred form of ondansetron crystalline solid form contains 1.3 to 1.5% of water by weight. In a substantially pure substance this corresponds to a hemi-hemihydrate form.

A further aspect of the present invention relates to a crystalline ondansetron base having a purity of at least 98% and being in the form of particles having a particle size not greater than 200 microns. Such a form is useful in making a variety of pharmaceutical dosage forms. Preferably the ondansetron particles have a size within the range of 0.1 to 100 microns, more preferably within the range of 0.1 to 63 microns.

Another aspect of the present invention relates to a composition comprising any of the above forms of ondansetron and a pharmaceutically acceptable excipient. The composition is preferably a unit dosage form for treating nausea and/or vomiting.

A further aspect of the invention relates to a process, which comprises neutralizing an acid addition salt of ondansetron to liberate ondansetron free base; and precipitating the ondansetron free base from a liquid media. Preferably this process produces form I ondansetron as is described in more detail hereinafter.

An additional aspect of the invention relates to a process which comprises dissolving ondansetron free base in a solvent and precipitating the dissolved ondansetron free base to form ondansetron having a melting point of greater than 240° C. Preferably this process produces form II ondansetron as is described in more detail hereinafter.

### BRIEF DESCRIPTION OF THE DRAWINGS

FIG. 1 shows a DSC curve for the material of Example 1.

FIG. 2 shows a XRPD pattern for the material of Example 1

FIG. 3 shows a DSC curve for the material of Example 1a.

FIG. 4 shows a XRPD pattern for the material of Example 1 a.

FIG. 5 shows a DSC curve for the material of Example 2.

FIG. 6 shows a XRPD pattern for the material of Example 2.

FIG. 7 shows a DSC curve for the material of Example 3.

FIG. 8 shows a XRPD pattern for the material of Example 3.

### DETAILED DESCRIPTION OF THE INVENTION

The present invention is based on the discovery that ondansetron base may be isolated in several solid state forms. Some of these forms differ from the form(s) recited in the above-mentioned patents in one or more respects. In general, the solid ondansetron forms of the present invention can be characterized by melting point, trace base or residue levels, and/or water content.

One form of ondansetron has a melting endotherm peak, i.e. a melting point, of at least 240° C., preferably within the range of 240° C. to 255° C. For purposes of the present invention, a melting endotherm peak is determined using differential scanning calorimetry (DSC) at a heating rate of 10° C./min. Other rates such as 5° C./min may be used. Preferably the ondansetron has two endotherm peaks wherein the first melting endotherm peak occurs at a temperature of 240° C. or greater. In this embodiment, both peaks generally occur within the temperature range of 240° C. to 255° C. Typically, the first and second endotherm peaks are within the range of 240° C.-249° C. and 249° C.-255° C., respectively. In a particularly preferred embodiment, the ondansetron solid form exhibits endotherm peaks at about 244° C. and 253° C.

Another ondansetron form can be characterized by the presence of a trace amount of a base or residue thereof which comprises an alkali metal, an amine, an ammonium, or an ion thereof. The base or its residue is preferably provided into the crystal/solid state form of the ondansetron as a result of forming the solid ondansetron by a neutralization process involving an ondansetron acid addition salt and a base. Thus, the base is preferably one that is sufficiently strong to neutralize an ondansetron acid addition salt and thereby liberate ondansetron free base. The residue of the base refers to a portion of a base, especially the post-neutralization product(s) thereof. Either the actual base, such as sodium hydroxide or a residue thereof such as a sodium ion, e.g. a sodium salt, can be present in the solid ondansetron form of this embodiment of the present invention. Preferably the base is an alkali metal-containing base, especially sodium or potassium hydroxide, more preferably sodium hydroxide. Typically the residue is all that is incorporated, i.e. a salt comprising sodium ion, potassium ion, etc. A “trace” amount as used herein means up to 1 wt %, preferably from 0.1 ppm to 1500 ppm, and more preferably is 1 ppm to 1000 ppm. Surprisingly, the ondansetron solid forms having a trace amount of the above-mentioned base or residue generally have a melting endotherm within the above-described known range, i.e. around 224° to 235° C.

Two specific forms of ondansetron are designated herein as form I and form II. Form I and form II have many different physical properties, such as in differential scanning calorimetry (DSC) or X-ray powder diffraction (XRPD) analysis, and thus may be identified or distinguished from one another by one or more properties.

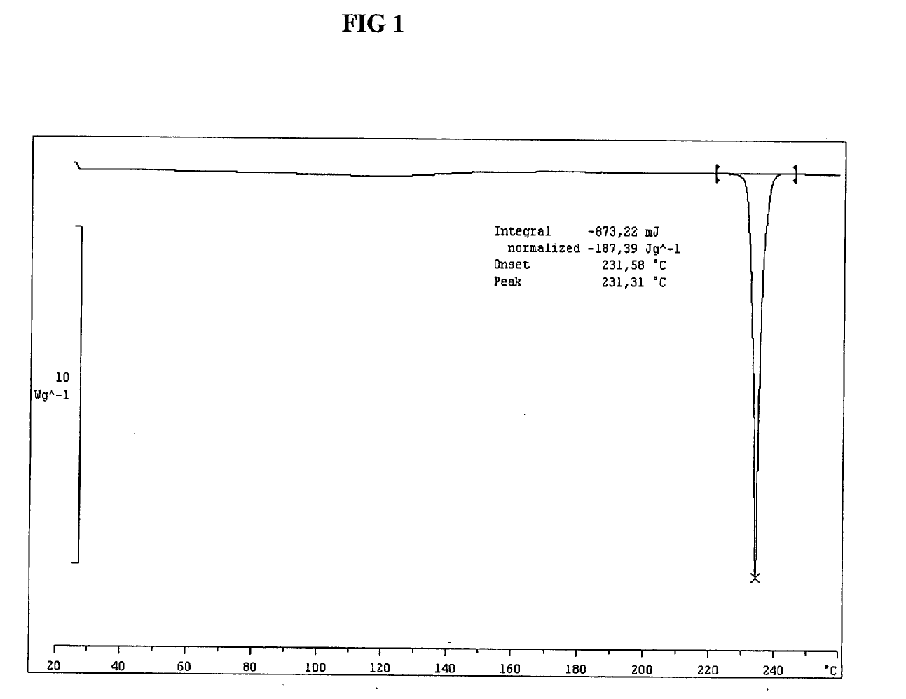

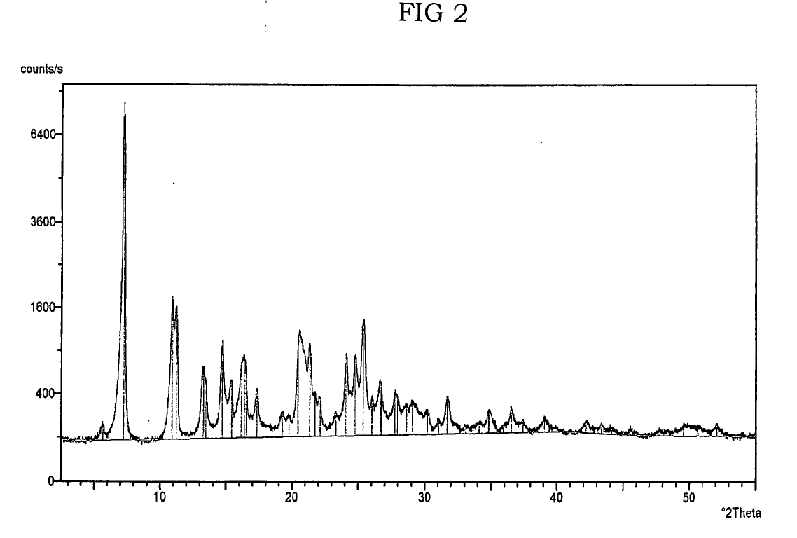

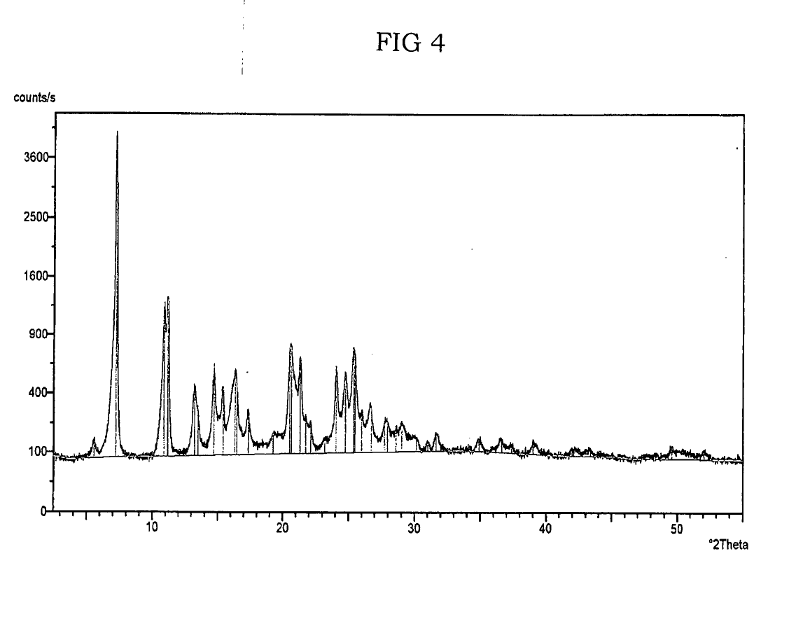

Form I ondansetron exhibits an X-RPD peak pattern that substantially corresponds to FIG. 2. “Substantially corresponds” is meant to cover variations/differences in curve or pattern that would not be understood by a worker skilled in the art to represent a difference in crystal structure, but rather differences in technique, sample preparation, etc. For example, the XRPD pattern shown in FIG. 4 substantially corresponds to the pattern shown in FIG. 2 even though it is not identical. The DSC curve exhibits a single sharp melting/degradation endotherm having a peak of about 224° C.-234° C.; their being some variation in the onset temperature and peak temperature. An example of a DSC scan for form I is shown in FIG. 1. Thermogravimetric analysis (TGA) reveals thermal degradation above 220° C.-230° C.

The form I ondansetron is sufficiently stable during storage at ambient and elevated temperatures. It is sensitive to a solvent-induced conversion into a form II defined below by slurrying in some polar solvents, e.g. in methanol or water, while it is inert to the same slurrying or solvent treatment in non-polar solvents.

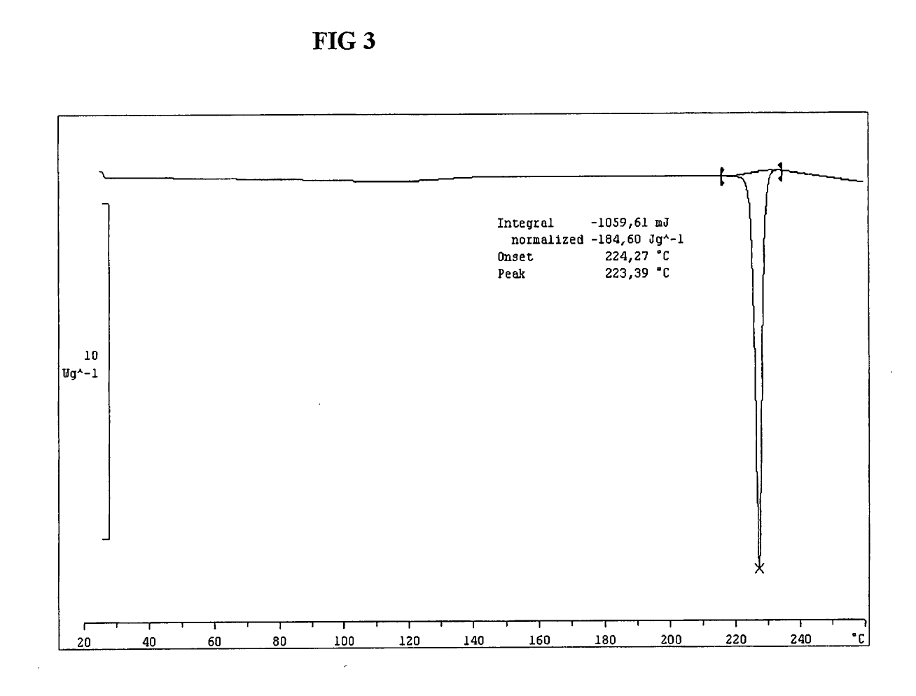

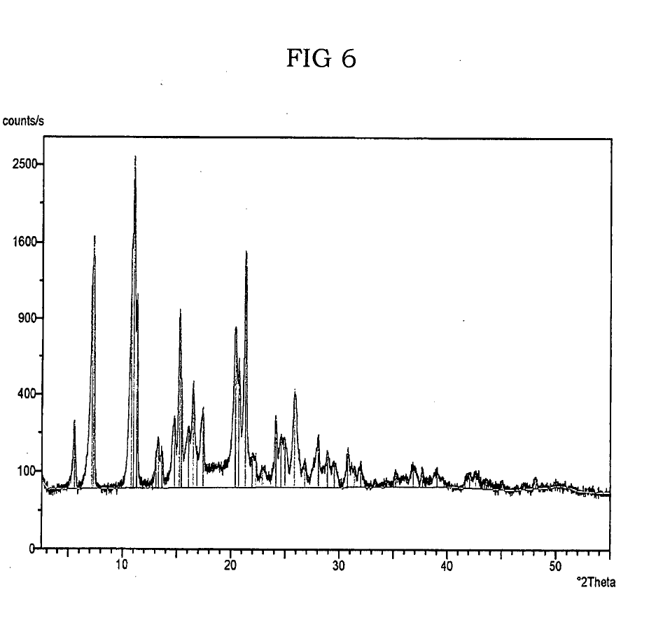

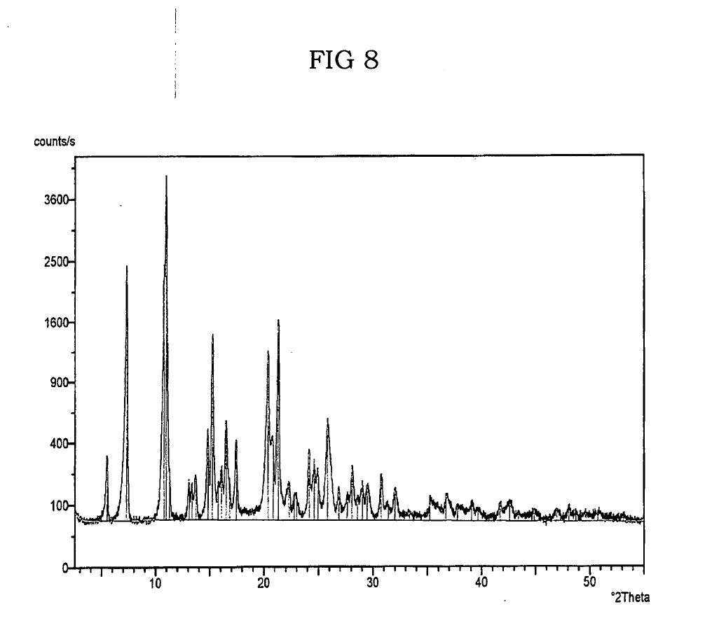

Form II ondansetron exhibits an XRPD peak pattern that substantially corresponds to FIG. 6. Similarly, the XRPD shown in FIG. 8 substantially corresponds to the pattern shown in FIG. 6. The DSC curve exhibits a first melting endotherm peak at 240° C. or greater and typically comprises two, usually overlapping, endotherms. Typically, these endotherm peaks are about 244° C. and 253° C., but may be also shifted to slightly lower temperatures. An example of a DSC curve for form II ondansetron is shown in FIG. 3. TGA shows thermal degradation above 240-250° C.

Form II is stable at room temperature when stored in closed vial, however a partial conversion to Form I was observed during prolonged storage at 40° C./75% relative humidity (RH). Form II is resistant to a solvent-inducing conversion to form I at ambient temperature.

Solid ondansetron base may exist in various states of hydration. An anhydrate form may be obtained by careful drying of the product, preferably under vacuum, at an enhanced temperature. Such anhydrate form comprises no or neglible amounts (less than 0.5%) of water.

After storage of the anhydrate form at humidity exceeding 10% RH, hydrates may be formed. A stable hydrated form comprise 1.3-1.5% of water, this corresponding to approximately 0.25 molar equivalent of water (a hemi-hemihydrate). Accordingly, another ondansetron form of the present invention is an ondansetron hydrate comprising ondansetron and water wherein the amount of water relative to ondansetron is within the range of 0.23-0.27 moles, more preferably 0.24-0.26 moles, per each one mole of ondansetron. Exposure of this or the anhydrated product to enhanced humidity (70% RH) results in a product having about 3% water content which corresponds to a hemihydrate (0.5 molar equivalent of water). Exposure to extreme humidity of about 90% or more leads to a monohydrate form of about 5% water. The most useful hydrated form of ondansetron base is the hemi-hemi hydrate as this is formed under most precipitation conditions and is stable. The above forms I or II are preferably hydrates containing 1.3-1.5% of water.

The solid ondansetron forms of the present invention as well as the prior art can be formed by precipitation. One process comprises neutralizing an acid addition salt of ondansetron to form ondansetron free base and precipitating the free base, sometimes referred to herein as the “neutralization process”. This process is generally advantageous for forming ondansetron form I. The acid addition salts of ondansetron include hydrochloride, hydrobromide, maleate, tartrate, mesylate, and tosylate, but are not limited thereto. Any suitable base, e.g., NaOH, KOH, amines, ammonium hydroxide, etc., for converting the ondansetron acid salt to ondansetron free base can be used to carry out neutralization.

In a first neutralization process, the solvent system is monophasic, i.e. it comprises a single solvent or a mixture of mutually miscible solvents, in which the resulting ondansetron base is only sparingly soluble and may thus precipitate and be separated from the remaining liquid. Advantageously, the solvent system is so selected that the starting ondansetron salt and the neutralization base are soluble in the solvent system, at least at an elevated temperature, but this is not required; i.e. a slurry of ondansetron acid addition salt can be used in the monophasic solvent system. Further, the solvent system should advantageously also dissolve the co-product of the reaction, i.e. the salt of the neutralizing base with the acid anion, so that the ondansetron base precipitates free from this co-product. The solvent should also preferably dissolve the side-products and impurities, particularly coloured impurities, which are eventually present in the starting ondansetron salt.

Suitable solvent systems comprise water and mixtures of water with water-miscible organic solvents such as lower aliphatic alcohol (methanol, ethanol), ketone (acetone, methyl isobutylketone) or cyclic ether (dioxan, tetrahydrofuran). In an advantageous mode, ondansetron salt is dissolved or suspended in one part of the solvent system and a solution or suspension of the neutralizing base in another part of the solvent system is added thereto portionwise until the reaction is completed. The composition of both parts of the solvent system may be identical or different. Completion of the neutralization reaction may be monitored, e.g. by measuring pH, the optimum value being of about 6 to about 9, more preferably 8-9.

The precipitation of the ondansetron free base from the monophasic solvent system, i.e. a liquid media, may be spontaneous or may be induced, e.g. by reducing the temperature of the solvent or by reducing the volume of the solution. This depends on the nature and amount of the solvent system and the proper mode of precipitation may be easily found by ordinary set of experiments. The temperature of contacting may be ambient, but, advantageously, the reaction mixture may be also heated, optionally up to reflux, and then cooled after the reaction is completed. In this way, a precipitate more easy to filter out may be formed. In another variant, an additional part of the solvent system, a contrasolvent, is added after the neutralization reaction is completed. The contrasolvent, which is a solvent in which the ondansetron base is insoluble, assists the precipitation by initiating the precipitation, increasing the yield of the precipitation, or both.

In the second mode of the neutralisation process, the solvent system is biphasic. The neutralisation reaction proceeds in a first, essentially aqueous phase and the product of the reaction is extracted into the second phase, which is immiscible with the first phase, while the rest of the reagents and the salt co-product remains in the first phase. After separation of the phases, the ondansetron base is precipitated from the solution in the second phase as described above.

Thus, the “liquid media” from which the liberated oridansetron free base is precipitated, can be the same liquid media that the neutralization reaction took place in, a modified solvent system, such as where solvent(s) are removed or contra-solvent(s) are added, etc., after neutralization, or an entirely different solvent system such as in a biphasic solvent system as described above.

The neutralization process is suitable for producing solid crystalline ondansetron having a trace amount of a base or residue as described above and/or for producing form I ondansetron. For producing form I ondansetron, a monophasic system comprising a mixture of water and ethanol in which ondansetron hydrochloride is used as the acid addition salt represents a preferred process.

Ondansetron solid forms can also be formed by precipitating dissolved ondansetron base. In particular, ondansetron base, such as isolated crude product, is dissolved in a suitable solvent, typically at elevated temperatures, and then the ondansetron is precipitated from the solution as an ondansetron solid form having a melting point of greater than 240° C. measured on DSC. This melting point refers to the first melting endotherm in the DSC analysis. The “dissolving” of ondansetron can be achieved by completing an ondansetron synthesis that results in the formation of ondansetron dissolved in the solvent as well as by dissolving solid ondansetron base in a solvent. Suitable solvents include methanol, ethanol, chloroform or ethyl acetate/methanol mixtures. The solution of ondansetron may optionally be treated or contacted with a suitable adsorption material, such as activated carbon, filtered, and cooled. The treatment preferably is carried out while the solution is hot, i.e. greater than 40° C. Ondansetron base precipitates after cooling and is separated by conventional methods such as filtration or centrifugation, and dried. Typically this form is form II ondansetron, particularly when the crystalline product separates out from the solution under elevated temperatures of about 40° C. and more. It is obtained also by a precipitation comprising contacting a solution of crude ondansetron base in a solvent, e.g. in methanol, with a contrasolvent such as n-heptane or water at ambient or diminished temperature. This process is also useful for removing colored impurities from isolated and/or crude ondansetron, especially when contacted with activated carbon.

While each of the above precipitation processes, optionally repeated one or more times, can provide for a purified or substantially pure ondansetron base, it has been discovered that the process of conversion of ondansetron base to a salt and reconversion of the salt back into precipitated ondansetron base (a “base-salt-base” process) is an efficient tool for purification of the original ondansetron base. Particularly, impurities resistant to purification by crystallization, e.g. colored impurities, may be removed this way. Crude or purified ondansetron base may be used for conversion into a suitable acid addition salt by a process employing a contact of ondansetron base with corresponding acid in a suitable solvent. The salt may be isolated in solid state. A preferred salt is ondansetron hydrochloride. Once the salt is formed, the neutralization process discussed above can be used to re-form ondansetron base in solid form.

In all of the above precipitation processes, the solid precipitate can be separated from the solution by conventional techniques, such as filtration, and is generally dried.

The above precipitation processes are also useful in producing substantially pure ondansetron is solid crystalline form. That is, ondansetron having a purity of at least 98%, preferably at least 99%, more preferably at least 99.5%, and even at least 99.9% purity, can be formed by any the processes. Such a degree of purity is advantageous in itself as ondansetron is intended to be used as a pharmaceutical.

It has been further discovered that ondansetron base having a particle size smaller than 200 microns (hereinafter “microcrystalline ondansetron”) is more suitable in making pharmaceutical formulations. For making liquid compositions, microcrystalline ondansetron dissolves more rapidly in the liquid medium. For making solid formulations, microcrystalline ondansetron produces more homogeneous compositions even when using processes that do not employ solvents for homogenization. Furthermore, the microcrystalline ondansetron releases more rapidly from the tablet composition.

Preferred particle sizes of microcrystalline ondansetron base for use in pharmaceutical final dosage forms is within the range of 0.1 to 200, more preferably 0.1 to 100, still more preferably 0.1 to 63 microns. At least 99% of the entire population of ondansetron particles should fall within these ranges. In some embodiments the particles are less than 20 microns, preferably less than 10 microns. For example, a population where 90% of the particles have a size of 2 microns or less. A representative ondansetron base population meets the following criterion:

| ≦250 μm | ≦63 μm | D (10) | D (50) | D (90) | |
|---|---|---|---|---|---|
| 100% | 100% | 0.5 μm | 0.8 μm | 1.6 μm | ² |

² measured in air by laser diffraction.

It is an advantage of the above neutralization process that such process allows for production of solid ondansetron base of the particle sizes defined above as “microcrystalline.” The particle size of the precipitated product may be controlled e.g. by the temperature regimen, nature of the solvent, concentration of the solution, etc. Proper production conditions may be found by an ordinary set of experiments.

Microcrystalline ondansetron base may be formed by crystallization of a crude ondansetron base from a solvent as well. In particular, it may be formed by mixing a hot solution of ondansetron base with a cold contrasolvent, whereby the temperature of contact is 20° C. or less, or by rapid cooling of an oversaturated solution of ondansetron base.

Furthermore, microcrystalline product may be formed by performing the precipitation or crystallization in ultrasonic bath. Ondansetron base of desired small particle size may also be obtained by micronizing in suitable micronization equipment known in the art, optionally in combination with sieving.

Ondansetron base, preferably microcrystalline ondansetron, may be formulated into various pharmaceutical compositions. In general a pharmaceutical composition, or a precursor thereof, comprises any of the above mentioned ondansetron base forms including the known ondansetron base in the above-recited purity or particle size, with a pharmaceutically acceptable excipient. The pharmaceutically acceptable excipient is not particularly limited and includes solid as well as liquid excipients and includes all of the excipients (categories and species) mentioned hereinafter with regard to the various compositional embodiments.

The composition may be formulated for parenteral administration, oral administration, rectal administration, transdermal administration and the like. The compositions for oral administration may be solid or liquid.

Liquid compositions for parenteral administration (injectable formulations) may be prepared from the ondansetron base, particularly from microcrystalline base, by dissolution. The dissolution may be advantageously performed by suspending the base in water, and adding a suitable pharmaceutically acceptable acid that forms a soluble salt. Suitable acid is hydrochloric acid. The acid is preferably used in an equimolar amount. The pH of the obtained solution may be imparted by an excess of an acid or by a pharmaceutically acceptable base. Preferred pH range is about 3-5. Furthermore, the composition may comprise a suitable buffer system to preserve the chosen pH range. An example of the buffer system is a citrate buffer, i.e. a mixture of citric acid and sodium citrate. In addition, the solution may comprise an isotonising agent and/or preservative. Suitable concentration of ondansetron in the liquid solution is from 0.1 to 10 mg/ml, preferably 2-4 mg/ml.

Liquid compositions for oral administration may be made for instance as disclosed in WO 96/15786, with the proviso that microcrystalline ondansetron base is the active ingredient and the solution also comprise a molar equivalent of a pharmaceutically acceptable acid.

Preferably, the pharmaceutical dosage forms formulated from the compositions of the invention comprise a unit dose of ondansetron, i.e. the therapeutically effective amount of ondansetron for a single dose administration. The preferred amount of the ondansetron base in the unit dose is from 0.1 to 150 mg, preferably 1, 2, 4, 8, 16, or 24 mg. The unit dose in a tablet form advantageously comprise one piece of the tablet but it also may comprise a divided tablets or one or more smaller tablets (minitablets) administered at the same time. In the last case, several smaller tablets may be advantageously filled into a gelatin capsule to form a unit dose. The unit dose of pellets in a capsule is advantageously contained in a single capsule. The unit dose of the injection solution is advantageously one vial. Solution for oral administration are preferentially packed in a multidose package, the unit dose being taken out by a calibrated vessel.

Solid compositions for oral administration may exhibit rapid, normal or extended release of the active substance from the composition. The solid pharmaceutical compositions comprising microcrystalline ondansetron base are preferably formulated into normal, immediate release tablets. Preferred tablet forms are disintegrable tablets. The tablets may comprise suitable inactive ingredients, i.e., excipients, such as filler(s)/diluent(s), binder(s), disintegrant(s), surfactant(s), lubricant(s) etc. They may be produced by any standard tabletting technique, e.g. by wet granulation, dry granulation or direct compression.

The tabletting methods that do not employ a solvent (“dry processes”) are preferable and the microcrystallinity of the active substance assures excellent homogenity of the mixture and good physical properties for tabletting.

The dry granulation procedure comprises mixing the solid excipients (except lubricants), compacting the mixture in a compactor (e.g. a roller compactor), milling the compacted mass, screening the milled granules, mixing with a lubricant and compressing the mixture into tablets.

The direct compression procedure comprises mixing the solid excipients and compressing the uniform mixture into tablets.

Ondansetron base may be also formulated by melt granulation, i.e. combining the ondansetron with a meltable functional excipient (e.g. glyceryl behenate) whereby upon heating a granulate is formed in suitable equipment. The granulae can be compressed into tablets, optionally with the addition of further excipients such as a lubricant.

Generally the amount of the ondansetron base in a tablet is from 1 to 10%, preferably 2-5%, based on the total weight of the tablet.

Ondansetron base may be also blended into compositions that are suitable for being formulated into pellets by pelletization techniques known in the art. A plurality of ondansetron base pellets comprising the single dose of ondansetron may be encapsulated into capsules made from pharmaceutically acceptable material, such as hard gelatin. In another mode, a plurality of pellets may be compressed together with suitable binders and disintegrants to a disintegrable tablet that, upon ingestion, decomposes and releases the pellets. In yet another mode, the plurality of pellets may be filled into a sachet.

Preferably, solid oral compositions comprising ondansetron base have the following release profile: more than 80% of the active is released in 30 minutes, most preferably in 15 minutes, when measured by a paddle method of Ph.Eur at 50 rpm in 0.01M HCl in a normal vessel. Alternatively, the same release results are achieved when measured in a peak vessel according to Van Kel. For such tablets, the microcrystalline ondansetron, as defined above, is especially suitable.

Tablets or pellets may be coated by a suitable coating, which may be a film coat (dissolvable in stomach environment) or an enteric coat (not dissolvable in stomach environment).

In particular, microcrystalline ondansetron can be formulated into rapidly disintegrable tablets, e.g. into tablets as described in U.S. Pat. No. 6,063,802.

The invention will be further explained by the following non-limiting examples.

### EXAMPLE 1

Process for Making Ondansetron Base by Neutralization (Form I)

680 g of ondansetron hydrochloride dihydrate was dissolved in 4000 ml of ethanol at reflux. A solution of 82 g of NaOH in 1000 ml of water was added. A solid was formed. 3000 ml of water was added and the mixture was cooled to ambient temperature. The solid was filtered off and washed with 2*500 ml of water. The solid was dried at 50° C. under vacuum for 2 days. The product exhibited the DSC curve shown in FIG. 1 and the XRPD pattern of FIG. 2.

Yield: 527 g (96%)

### Example 1A

### Ondansetron Base by Neutralization

80 g of ondansetron hydrochloride dihydrate was suspended in 500 ml of ethanol and heated to reflux until a clear solution was obtained. To this solution, 250 ml of a 1 M NaOH solution was added. During addition a solid started to form. 250 ml of water was added and the mixture was slowly cooled to room temperature. The mixture was cooled to 10-15° C. and the solid was filtered off. The solid was washed with 2×200 ml of water. After drying in a vacuumoven at 40° C. for 3 days. 59.3 g of a white solid was obtained. The product exhibited the DSC curve shown in FIG. 3 and the XRPD pattern of FIG. 4.

Yield: 59.3 g (92%)

### EXAMPLE 2

### Ondansetron Base Form II

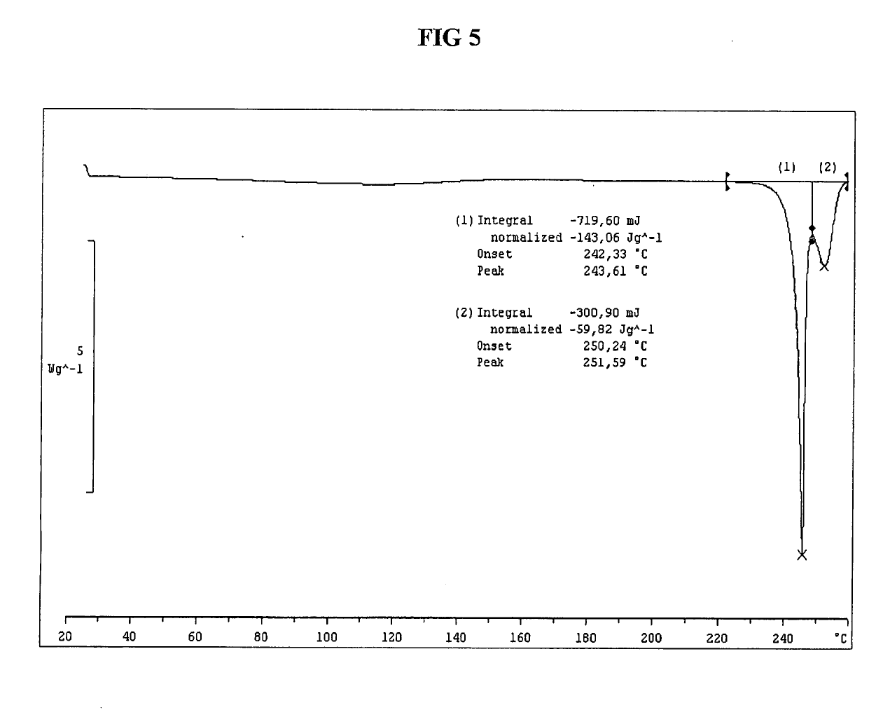

3.3 gram of ondansetron base and 60 ml methanol were transferred into a 3 neck glass flask of 100 ml. The suspension was refluxed for approximately 10 minutes and 20 ml of methanol was subsequently added to the suspension. The suspension was refluxed again. After addition of next 17 ml of methanol, reflux was maintained until a clear solution was obtained. The solution was left in the oil bath and allowed to cool under stirring. During cooling the temperature was measured and a cooling rate of approximately 1° C./1.5 minutes was observed. Rapid crystallization of thin needles, agglomerated in flocks, occurred at T=53° C. The cooling procedure was continued until approximately 31° C. The crystals were filtered off on a p3-glass filter and washed with methanol. The sample was dried at room temperature under vacuum overnight. The yield was 2.41 g (approx. 73%) of ondansetron base form II. The product exhibited the DSC curve shown in FIG. 5 and the XRPD pattern of FIG. 6.

### EXAMPLE 3

### Ondansetron Base Form II

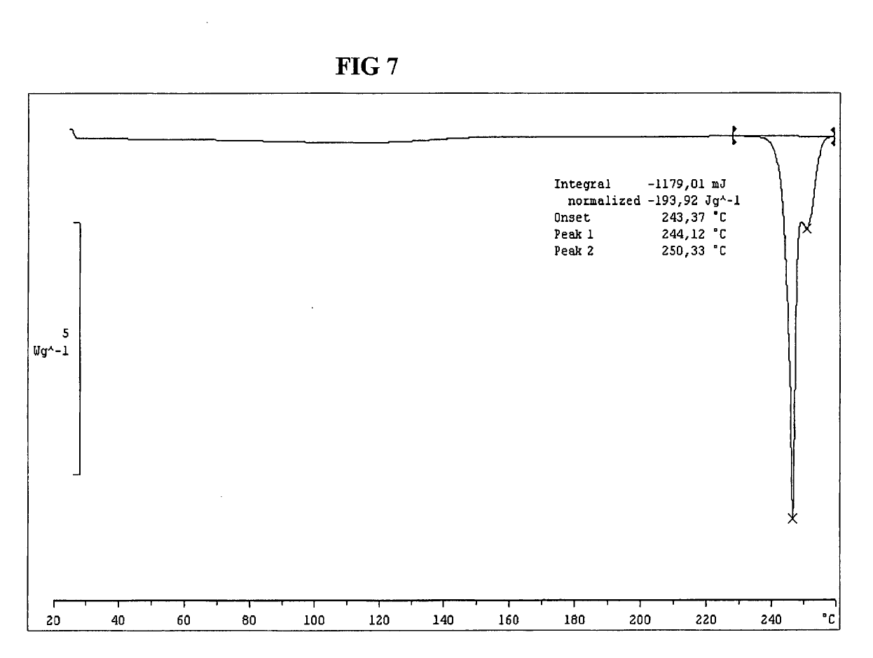

3.3 gram of ondansetron base and 110 ml of methanol were transferred into a 3 neck glass flask of 250 ml. The suspension was slowly heated in an oil bath to reflux and stirred with a magnetic stirrer and stirrer device. The solid slowly dissolved within 30 minutes. The solution was then warm filtered off on a p3-glass filter into a round bottomed flask of 250 ml. During filtration a few crystals of solid appeared. To the filtered solution 5 ml of methanol was added to compensate for possible evaporation of solvent during filtration. The solution was stirred and refluxed for approximately 15 minutes. Then the solution was cooled down slowly by stepwise lowering the temperature of the oil bath. Complete crystallization has occurred after 2.5 hours (white cake formed). To the content of the flask added 5 ml of methanol. The suspension was stirred and refluxed for 30 minutes. The obtained clear solution was then slowly cooled down by cooling down the oil bath. When the solution stopped refluxing, a few mg of Ondansetron form II was added as seed. The solution was then cooled down for a few more degrees. After about 10 minutes, fine particles appeared and the solution was cooled down for a few more degrees. After 10 more minutes, crystallization of fine needles occurred, soon agglomerating to flocks. Prolonged crystallization occurred within 40 minutes. The crystals were then filtered off on a p3-glass filter and washed with methanol. The sample was dried at room temperature under vacuum overnight. The yield was 2.41 g (approx. 73%) of form II. The product exhibited the DSC curve shown in FIG. 7 and the XRPD pattern of FIG. 8.

Peak table of the most pronounced signals from the above XRPD patterns

| Ex. 1a: Form I | Ex. 1: Form I | Ex. 3: Form II |
|---|---|---|
| | | 5.57 |
| 7.25 | 7.24 | 7.35 |
| 10.90 | 10.92 | 10.83 |
| | | 11.06 |
| 11.21 | 11.26 | |
| 13.24 | 13.28 | 13.12 |
| | | 13.36 |
| | | 13.67 |
| 14.73 | 14.72 | 14.83 |
| 15.42 | 15.43 | 15.32 |
| 16.46 | 16.47 | 16.55 |
| 17.35 | 17.24 | 17.45 |
| | | 24.65 |
| 24.74 | 24.76 | 24.96 |
| 25.37 | 25.35 | 25.86 |

### EXAMPLE 4

Injectable Solution of Ondansetron Base (2 mg/ml Injection):

Composition of the Injection

| | per ml | |
|---|---|---|
| Active ingredient | | |
| Ondansetron base | 2.00 | mg |
| Excipients | | |
| Citric Acid monohydrate | 0.5 | mg |
| Sodium citrate dihydrate | 0.25 | mg |
| Sodium chloride | 9.0 | mg |
| Hydrochloric acid solution 1M | 6.8 | μl |
| Hydrochloric acid solution 1M, | q.s. ad | |
| | pH 3-5 | |
| Sodium hydroxide solution 1M | q.s. ad | |
| | pH 3-5 | |
| Nitrogen/Argon | q.s. | |
| Water for injections | ad 1.0 | ml |

Process for Making Injections

Various orders for mixing the components in formulation the solution were used. In the formulations, pH was adjusted by HCl or NaOH to the desired level prior to adding the last amount of water.

| Variant | Order of mixing |
|---|---|
| A | a) 80% water, citric acid, citrate, NaCl, |
| | b) ondansetron base, |
| | c) HCl, |
| | d) 20% water |
| B | a) 80% water, HCl, |
| | b) ondansetron base, |
| | c) citric acid, citrate, NaCl, |
| | d) 20% water |
| C | a) 80% water, citric acid, citrate, NaCl, |
| | b) HCl, |
| | c) ondansetron base, |
| | d) water |
| D | a) 80% water, HCl, citric acid, |
| | b) ondansetron base |
| | c) citrate, NaCl, |
| | d) water |

### EXAMPLE 5

### Tablets of Ondansetron Base

Compositon per 1 g of Tablet Core:

| | |
|---|---|
| Ondansetron base | 32 mg |
| Lactose anhydrous | 665 mg |
| Microcrystalline cellulose | 250 mg |
| Preg. maize starch | 50 mg |
| Magnesium stearate | 5 mg |

Process:

1. Sieve the ondansetron base trough a 500 μm sieve, sieve the excipients through a 850 μm sieve.

2. Mix the ondansetron base with half the amount of lactose for 5 minutes in a free fall mixer.

3. Add the remaining part of the lactose and mix another 5 minutes

4. Add the MCC and the pregelatinized maize starch and mix for 15 minutes.

5. Add the Mg Stearate and mix for 3 minutes 6. Press 4 mg—containing tablets using a Korsch EKO excentric press

### Example 6

### Tablets of Ondansetron Base

Composition per 1 g of Tablet Core:

| | |
|---|---|
| Ondansetron base | 32 mg |
| Lactose anhydrous | 657 mg |
| Microcrystalline cellulose | 251 mg |
| Preg. maize starch | 50 mg |
| Magnesium stearate | 5 mg |
| Talc | 5 mg |

Process:

1. Sieve the ondansetron base trough a 500 μm sieve, sieve the excipients through a 850 μm sieve.

2. Mix the ondansetron base with half the amount of lactose for 5 minutes in a free fall mixer

3. Add the remaining part of the lactose and mix another 5 minutes

4. Add the MCC and the pregelatinized maize starch and mix for 15 minutes.

5. Add the Mg Stearate and the Talc and mix for 3 minutes.

6. Press 4 mg and 8 mg—containing tablets using a Korsch EKO excentric press

### Example 7

### Tablets of Ondansetron

Composition per 1 g of Tablet Core:

| | |
|---|---|
| Ondansetron base | 32 mg |
| Lactose anhydrous | 683 mg |
| Microcrystalline cellulose | 260 mg |
| Sodium Starch Glycolate | 20 mg |
| Magnesium stearate | 5 mg |

Process:

1. Sieve the ondansetron base trough a 500 μm sieve, sieve the excipients through a 850 μm sieve.

2. Mix the ondansetron base with half the amount of lactose for 5 minutes in a free fall mixer

3. Add the remaining part of the lactose and mix another 5 minutes

4. Add the MCC and the Sodium starch glycolate and mix for 15 minutes

5. Add the Mg Stearate and mix for 3 minutes.

6. Press 4 mg and 8 mg—containing tablets using a Korsch EKO excentric press

### Example 8

### Tablets of Ondansetron

Composition per 1 g of Tablet Core:

| | |
|---|---|
| Ondansetron base | 32 mg |
| Lactose anhydrous | 683 mg |
| Microcrystalline cellulose | 250 mg |
| Sodium Starch Glycolate | 20 mg |
| Magnesium stearate | 5 mg |
| Talc | 10 mg |

Process:

1. Mix ondansetron base and ¼ of the amount of lactose for 5 minutes in a turbula mixer and sieve the pre-blend through a 500 μm sieve.

2. Transfer the sieved pre-blend into the turbula. Sieve (500 μm) and add ¼ of the lactose and mix for 5 min.

3. Transfer the blend into a free fall mixer. Sieve (500 μm) and add the remaining part of the lactose and mix for 5 minutes

4. Sieve (500 μm) and add the MCC and the SSG and mix for 15 minutes

5. Sieve (500 μm), add the magnesium stearate and talc, and mix for 3 minutes

6. Press 8 mg—containing tablets using a Korsch EKO excentric press

The invention having been described, it will be readily apparent to those skilled in the art that further changes and modifications in actual implementation of the concepts and embodiments described herein can easily be made or may be learned by practice of the invention, without departing from the spirit and scope of the invention as defined by the following claims.

What is claimed is:

1. A solid crystalline ondansetron having at least one of the following characteristics:
a melting endotherm peak greater than or equal to 240° C.;
a trace amount of a base or residue thereof comprising an alkali metal, an amine, an ammonium or an ion thereof; or
a water content of 1.3 to 1.5 wt %.

2. The ondansetron according to claim 1, wherein said ondansetron has a first melting endotherm peak greater than or equal to 240° C.

3. The ondansetron according to claim 1, which is form II ondansetron.

4. The ondansetron according to claim 3, which was formed by precipitating said solid crystalline ondansetron from a solution at a temperature greater than or equal to 40° C.

5. The ondansetron according to claim 1, wherein said ondansetron has a powder x-ray diffraction pattern that substantially corresponds to FIG. 6.

6. The ondansetron according to claim 1, wherein said ondansetron contains a trace amount of a base or residue thereof which comprises an alkali metal, an amine, an ammonium, or an ion thereof.

7. The ondansetron according to claim 6, wherein said base or residue is contained in an amount of from 1 ppm to 1000 ppm.

8. The ondansetron according to claim 7, wherein said base or residue comprises an ion of an alkali metal.

9. The ondansetron according to claim 8, wherein said alkali metal is sodium.

10. The ondansetron according to claim 1, wherein said ondansetron is a hydrate.

11. The ondansetron according to claim 10, comprising ondansetron and water wherein the amount of water relative to ondansetron is within the range of 0.23-0.27 moles per each one mole of ondansetron.

12. The ondansetron according to claim 11, wherein the amount of water is within the range of 0.24 to 0.26 moles per each one mole of ondansetron.

13. The ondansetron according to claim 1, wherein said ondansetron has a water content of 1.3 to 1.5 wt %.

14. The ondansetron according to claim 2, wherein said ondansetron has a water content of 1.3 to 1.5 wt %.

15. A crystalline ondansetron base having a purity of at least 98% and being in the form of particles wherein at least 99% of the particles have a particle size within the range of 0.1 to 63 microns.

16. The ondansetron according to claim 15; wherein at least 99% of the particles have a particle size less than 10 microns.

17. The ondansetron according to claim 15, wherein 90% said particles have a size of 2 microns or less.

18. A composition comprising the ondansetron according to claim 1 and a pharmaceutically acceptable excipient.

19. The composition according to claim 18, wherein said composition is a unit dose form.

20. The composition according to claim 19, wherein said ondansetron is contained in an amount of 0.1 to 150 mg.

21. The composition according to claim 20, wherein said amount of ondansetron is 1, 2, 4, 8, 16, or 24 mg.

22. A process, which comprises:
neutralizing an acid addition salt of ondansetron to liberate ondansetron free base; and
precipitating said ondansetron free base from a liquid media.

23. The process according to claim 22, wherein said precipitated ondansetron is form I ondansetron.

24. The process according to claim 23, wherein said neutralization and precipitation is carried out in one liquid phase.

25. The process according to claim 24, wherein said liquid phase comprises a solvent selected from the group consisting of a C₁-C₆ aliphatic alcohol, C₁-C₆ aliphatic ketone, C₃-C₆ cyclic ether, water, or mixtures of two or more thereof.

26. The process according to claim 24, wherein a contra-solvent is added to said liquid phase after said neutralization step in order to assist said precipitation step.

27. The process according to claim 23, wherein said neutralization is carried out in a first liquid phase and said precipitation is carried out in a second liquid phase.

28. The process according to claim 23, wherein said precipitated ondansetron has an average particle size of 20 microns or less.

29. The process according to claim 23, which further comprises converting crude ondansetron base into said acid addition salt of ondansetron, optionally followed by isolation of said salt before said neutralizing step.

30. A process, which comprises dissolving ondansetron free base in a solvent and precipitating said dissolved ondansetron free base to form ondansetron having a melting endotherm peak of greater than or equal to 240° C.

31. The process according to claim 30, wherein said precipitated ondansetron is form II ondansetron.

32. The process according to claim 31, wherein said precipitation occurs at one or more temperatures greater than 40° C.

33. The process according to claim 31, which further comprises contacting said solvent containing said dissolved ondansetron with activated charcoal prior to said precipitating step.
